(19) United States
(12) Patent Application Publication
(10) Pub. No.: US 2014/0180260 A1
(43) Pub. Date: Jun. 26, 2014

(54) METHODS RELATED TO A BAGLESS CATHETER

(71) Applicant: Edward J. MUELLER, Helotes, TX (US)

(72) Inventor: Edward J. MUELLER, Helotes, TX (US)

(21) Appl. No.: 14/083,902

(22) Filed: Nov. 19, 2013

Related U.S. Application Data
(60) Provisional application No. 61/728,307, filed on Nov. 20, 2012.

(51) Int. Cl. A61M 25/00

(52) U.S. Cl. CPC A61M 25/0017; A61M 25/0075
USPC 604/544

(57) ABSTRACT

A bagless catheter. At least some of the illustrative embodiments are methods including: selectively storing urine in a bladder of a patient by: inserting a first end of a tube through a urethra and into the bladder of the patient, wherein a second end of the tube, opposite the first end, is coupled to a valve; accumulating urine in the bladder during a time period when the valve is in a closed position; discharging urine into a waste receptacle, the urine discharging from the second end of the tube through air and into the waste receptacle, the discharging when the valve is in an open position; and closing the valve.

### CROSS-REFERENCE TO RELATED APPLICATIONS

This application claims the benefit of Provisional Application Ser. No. 61/728,307 filed Nov. 20, 2012, titled “Method and System for a Bagless Catheter Valve,” which provisional application is incorporated by reference herein as if reproduced in full below.

### BACKGROUND

Catheters provide patients with a way to drain urine, however, catheter systems can be messy, inconvenient, embarrassing, and potentially difficult to operate. Thus, any method of easily and discretely draining urine from a patient's bladder would provide a competitive advantage in the marketplace.

### BRIEF DESCRIPTION OF THE DRAWINGS

For a detailed description of various embodiments, reference will now be made to the accompanying drawings in which:

FIG. 1 shows a catheter system coupled to a bag;

FIG. 2 shows an elevation, partial cross-sectional, view of a bagless catheter system in accordance with at least some embodiments;

FIG. 3 shows an elevation view of a closed bagless catheter valve according to some embodiments;

FIG. 4 shows an elevation view of an open bagless catheter valve according to some embodiments;

FIG. 5 shows, in block diagram form, a method in accordance with at least some embodiments; and

FIG. 6 shows, in block diagram for , a method in accordance with at least some embodiments.

### NOTATION AND NOMENCLATURE

Certain terms are used throughout the following description and claims to refer to particular system components. As one skilled in the art will appreciate, different companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Waste receptacle” shall mean a container which is not a bag and which is neither coupled to the patient nor coupled to a catheter.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various embodiments are directed the insertion of and use of a bagless catheter. In particular, various embodiments are directed to selectively storing urine naturally in a human bladder, and subsequently draining the urine directly into a toilet, urinal, bed pan or other non-coupled, non-bag waste receptacle, the draining by way of a catheter tube coupled to a valve mechanism. The specification first turns to a discussion of the related-art systems to orient the reader.

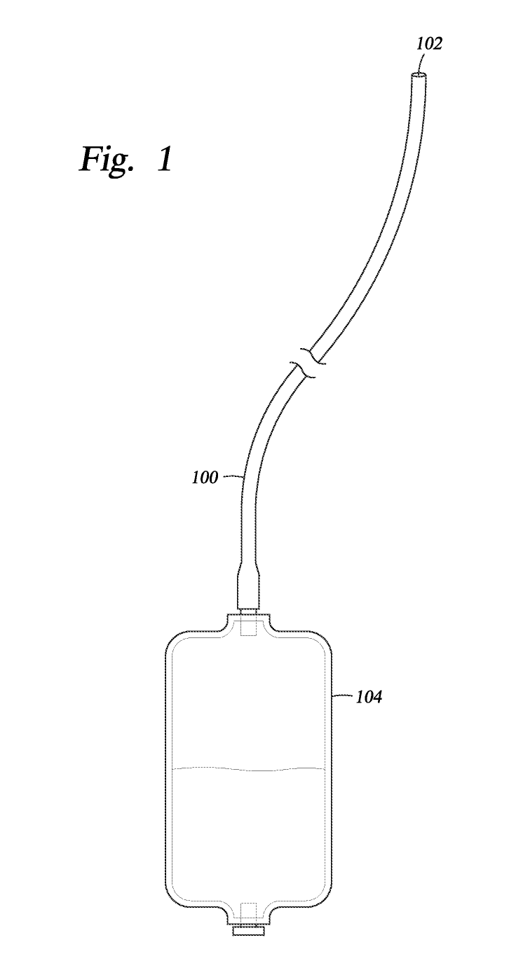

FIG. 1 shows a catheter system coupled to a urine collection bag 104 in accordance with a related-art system. In some cases, a catheter is inserted directly into a patient's bladder directly through the abdomen at a location just above the pubic bone. The catheter is then connected to extension tubing which connects to a urine collection bag 104 residing outside of the patient's body. In the related art, patient's urine drains from the bladder as the urine is produced. More specifically, as a patient's kidneys secrete urine into the bladder, the urine is immediately discharged out of the patient's body through the end 102 of catheter tubing located in the patient's bladder and into the bag 104.

The bag 104, as well as a portion of the tubing residing outside of the patient's body, is attached to the patient's leg (e.g. thigh or calf) in order to collect the urine as it drains from the bladder. The bag of collected urine remains attached to the patient until the bag 104 is emptied or otherwise discarded. In other cases, the tubing and the bag are coupled to the patient's abdomen. In still other cases, the tubing is longer so that the bag 104 can be attached to the side of a bed or a wheelchair.

Regardless of the exact placement of the tubing and the bag, the bag must be positioned and adjusted to avoid any blockage (e.g., bends or kinks in the tubing). Furthermore, the patient must be constantly aware of keeping the bag below the level of the bladder to prevent the backflow of urine), as a backflow of urine into the bladder may cause recurrent urinary tract infections

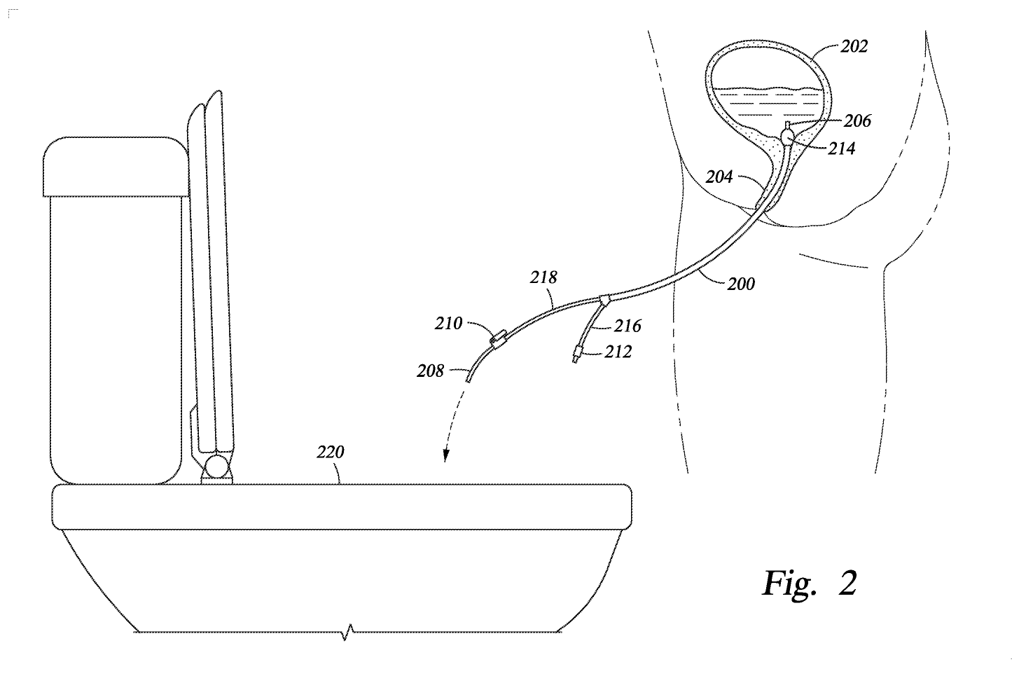

FIG. 2 shows, by contrast, a bagless catheter inserted through patient's urethra and into the patient's bladder in accordance with at least some embodiments. A bagless catheter, unlike related-art systems, mimics normal bladder function by using a patient's bladder to store urine. Furthermore, the bagless catheter helps a patient maintain normal bladder function during the period of catheterization. Through a valve system coupled to the bagless catheter, a patient may “urinate” at an appropriate moment and in an appropriate location.

In particular, a bagless catheter system is comprised, in part, of a length of flexible tube 200. Catheter tubing is well-known in the art, and may be comprised of a variety of different materials (e.g., latex, polyurethane, silicone). The catheter tube 200 may be selected from a variety of different thicknesses in order to accommodate the natural variations of human anatomy and to personalize the catheter system to the specific patient.

Through a process called catheterization, one end 206 of a catheter tube 200 is inserted through a patient's urethra 204 into the patient's bladder 202. The opposite end of the catheter tube 202 (i.e., the end of the tube 202 which is not inside the bladder), forks into two tube channels, or lumens 216 and 218. In one embodiment, lumen 216 comprises a balloon port 212 which controls inflation of a balloon 214 coupled to the end 206 of tube 200. Balloon 214 is in a deflated stated during the time when the end 206 of the catheter system is inserted into the bladder; however, once the catheter tubing is correctly placed inside the patient's bladder, balloon 214 is inflated in order to reduce the likelihood of the catheter tubing from accidentally slipping out of the bladder. More specifically, in order to inflate the balloon 214, a syringe filled with sterile water is coupled to the balloon port 212 (such as by way of a Luer-Lok™ connection). Using positive pressure, the sterile water is pushed out of the syringe through balloon port 212 and into balloon 214, filling balloon 214 to a predetermined volume.

In one embodiment, lumen 218 terminates in an open channel at end 208 (i.e., open to the air) and comprises a valve mechanism 210 which is controlled by a lever. When the patient wants to drain urine from the bladder, the patient may manipulate the lever to open the valve, thus enabling urine drainage in a way that Mimics the natural evacuation of urine. Upon closing the valve mechanism 210, the patient's urine is accumulated and stored in the patient's bladder 102 until such time as the patient wishes to discharge the urine.

More specifically, when the patient wishes to urinate, the patient opens the valve to unseal the channel provided by lumen 218 (wherein the open channel terminates at end 208). The urine in the bladder 102 flows into the tube 200 by way of end 206, and drains through the valve mechanism 210 and out of end 208. Once the urine is discharged out of the tube 200 through end 208, the urine falls through the air into a toilet 220 or other waste receptacle, such as a bed pan or urinal, thus completely obviating the need for a storage bag coupled to the tubing. Furthermore, because the urine exits the bagless catheter system upon discharge, and because there is no external storage of the urine with a connected pathway back into the bladder, there is no risk of backflow of the patient's urine, and thus there is a decreased risk of infection or other complications.

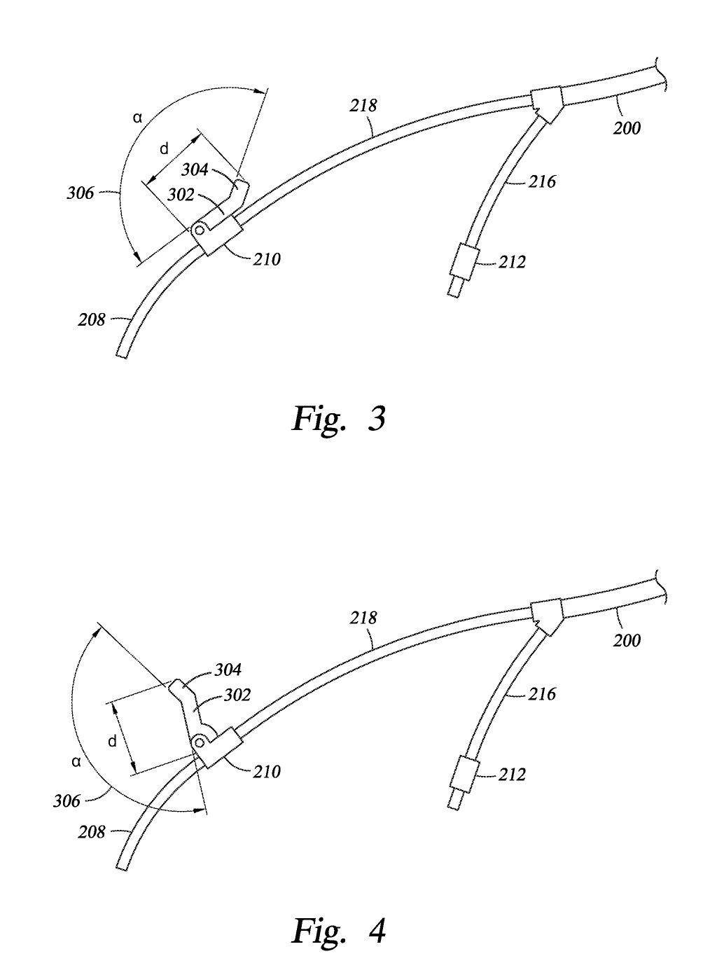

The specification now turns to a more detailed discussion of an example valve mechanism 210. FIG. 3 shows a portion of a bagless catheter tube 200 in a closed position in accordance with at least some embodiments. In particular, and as discussed previously with reference to FIG. 2, lumen 218 comprises valve mechanism 210. Valve mechanism, in turn, comprises lever 302. When the example lever 302 is in a closed position, the lever 302 may be parallel to the length of lumen 218. When lever 302 is in a closed position, the valve mechanism 210 is closed, enabling urine to accumulate in the patient's bladder, and preventing urine from discharging from the bladder. When the patient's bladder is full and/or the patient has an urge to urinate, the patient opens the valve 302 in order to enable drainage of the stored urine into a toilet or other waste receptacle, such as a bed pan or urinal. Example valve mechanism 210 is opened by rotating lever 302 to a perpendicular position, as shown in FIG. 4, but other types may be equivalently used. It should be noted that, regardless of the type of waste receptacle into which the urine is discharged, the waste receptacle is not coupled to any part of the bagless catheter system; in other words, urine does not drain into a bag coupled to either the catheter tubing or to the patient, but exits into the air before collecting in a detached waste receptacle.

FIG. 4 shows the bagless catheter with lever 302 in an open position (e.g., perpendicular to the length of lumen 218). In one embodiment, the patient may have a condition that makes operating the lever difficult; for example, a lack of dexterity and/or strength. Thus, the lever 302 may be designed to have a bent portion 304 at the distal end, the bent portion bent so as to create angle 306 “α.” Bent portion 304 enables a person operating the lever 302 to better grip the lever, thus enabling easier rotation. Angle 306 “α” may be any angle which enables a patient to place at least one finger, or other instrument, under lever 302 for rotational leverage.

In addition, the lever 302 may be of a certain length “d.” Length “d” is of a sufficient measurement such that it enables a person operating the lever 302 to apply less force to the lever in rotating it to a perpendicular position, thus enabling opening of the valve mechanism 210. When the patient, or caregiver, desires to close the valve, the bent edge may provide a source of leverage for the lever to be rotated back to a parallel position. Furthermore, as with opening the valve, the length “d” of the valve lever enables less force to be used in rotating the valve lever back to a parallel position.

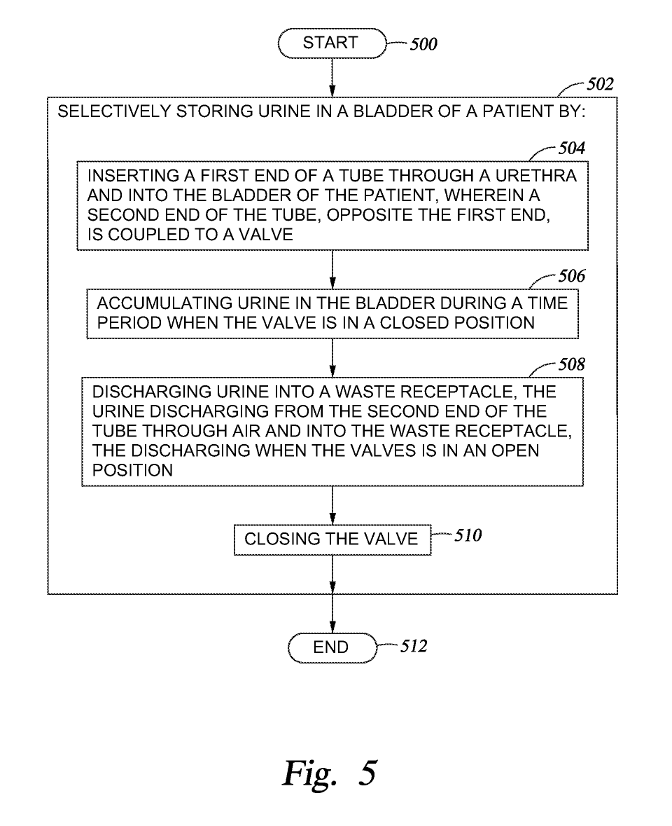

FIG. 5 shows a method in accordance with at least some embodiments. In particular, the method starts (block 500) and comprises selectively storing urine in a bladder of a patient (block 502) by; inserting a first end of a tube through a urethra and into the bladder of the patient, wherein a second end of the tube, opposite the first end, is coupled to a valve (block 504); accumulating urine in the bladder during a time period when the valve is in a closed position (block 506); discharging urine into a waste receptacle, the urine discharging from the second end of the tube through air and into the waste receptacle, the discharging when the valve is in an open position (block 508); and closing the valve (block 510). Thereafter, the method ends (block 512).

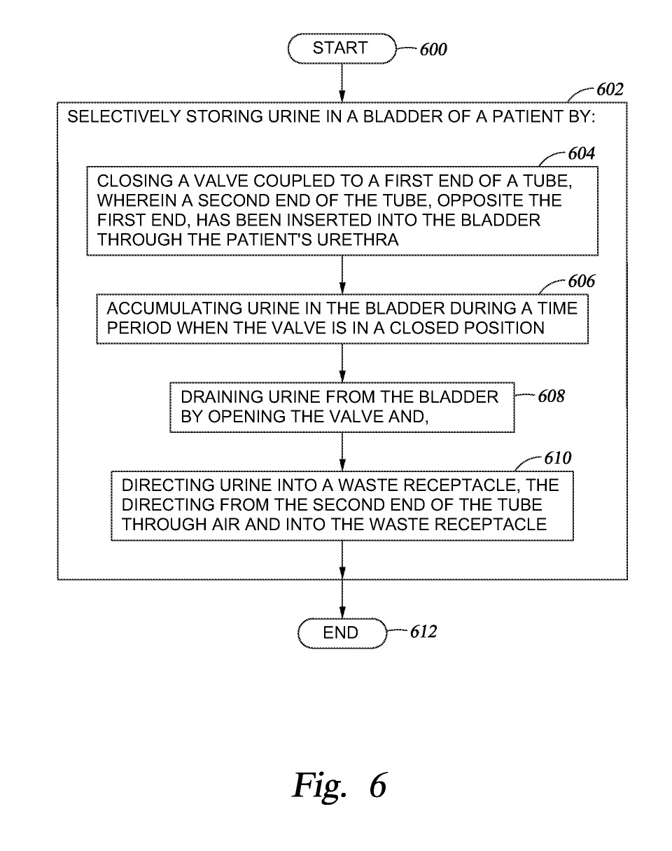

FIG. 6 shows a method in accordance with at least some embodiments. In particular, the method starts (block 600) by: selectively storing urine in a bladder of a patient (block 602) by: closing a valve coupled to a first end of a tube, wherein a second end of the tube, opposite the first end, has been inserted into the bladder through the patient's urethra (block 604); accumulating urine in the bladder during periods of time when the valve is dosed (block 606); draining urine from the bladder by opening the valve (block 608); directing urine into a waste receptacle, the directing from the second end of the tube through air and into the waste receptacle (block 610). Thereafter, the method ends (block 612).

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications. References to “one embodiment”, “an embodiment”, “a particular embodiment”, “some embodiments”, “various embodiments”, and “example embodiments” indicate that a particular element or characteristic is included in at least one embodiment of the invention. Although the illustrative phrases may appear in various places, these do not necessarily refer to the same embodiment.

What is claimed is:

1. A method comprising:
selectively storing urine in a bladder of a patient by:
inserting a first end of a tube through a urethra and into the bladder of the patient, wherein a second end of the tube, opposite the first end, is coupled to a valve;
accumulating urine in the bladder during a time period when the valve is in a closed position:
discharging urine into a waste receptacle, the urine discharging from the second end of the tube through air and into the waste receptacle, the discharging when the valve is in an open position; and
closing the valve.

2. The method of claim 1 further comprising sealing the valve by way of adjusting a valve member.

3. The method of claim 2 wherein adjusting the valve member further comprises the patient adjusting a valve level into a parallel position relative to the length of the valve member.

4. The method of claim 1 further comprising unsealing the valve by way of adjusting a valve lever into a non-parallel position relative to the length of the valve.

5. The method of claim 1 where in discharging urine further comprises discharging urine into at least one selected from the group consisting of: a toilet; a bed pan; and a urinal.

6. A method comprising:
selectively storing urine in a bladder of a patient by:
closing a valve coupled to a first end of a tube, wherein a second end of the tube, opposite the first end, has been inserted into the bladder through the patient's urethra;
accumulating urine in the bladder during periods of time when the valve is closed;
draining urine from the bladder by opening the valve; and
directing urine into a waste receptacle, the directing from the second end of the tube through air and into the waste receptacle.

7. The method of claim 6 further comprising dosing the valve by way of adjusting a valve member.

8. The method of claim 7 wherein adjusting the valve member further comprises the patient adjusting a valve lever into a parallel position relative to the length of the valve.

9. The method of claim 6 further comprising opening the valve by way of adjusting a valve lever into a non-parallel position relative to the length of the valve.

10. The method of claim 6 where in discharging urine further comprises discharging urine into at least one selected from the group consisting of: a toilet; a bed pan; and a urinal.
